(19) United States
(12) Patent Application Publication
(10) Pub. No.: US 2020/0054199 A1
(43) Pub. Date: Feb. 20, 2020

(54) PACKAGE

(71) Applicant: OLYMPUS CORPORATION, Tokyo (JP)

(72) Inventors: Shu KAMBE, Tokyo (JP); Kosuke KISHI, Tokyo (JP)

(73) Assignee: OLYMPUS CORPORATION, Tokyo (JP)

(21) Appl. No.: 16/661,070

(22) Filed: Oct. 23, 2019

Related U.S. Application Data
(63) Continuation of application No. PCT/JP2017/016905, filed on Apr. 28, 2017.

(51) Int. Cl. A61B 1/00

(52) U.S. Cl. CPC A61B 1/00144; H01R 13/5213

(57) ABSTRACT

A package is composed of a resin molded body packaging a manipulator of a manipulator system including a base member having a target portion on an upper surface, and the manipulator having a connecting portion detachably connected to the target portion. The package includes a cover portion having a size capable of covering at least a portion of the upper surface of the base member, and a forming portion having easy-cut lines provided in a portion of the cover portion. The forming portion is configured such that by cutting the forming portion along the lines, it is possible to form, at a position corresponding to the target portion when the covering portion is disposed at a position covering the upper surface, an opening portion penetrating a predetermined region including the target portion in the plate thickness direction and wall portions rising upward from an edge of the opening portion.

### CROSS-REFERENCE TO RELATED APPLICATIONS

This is a continuation of International Application PCT/JP2017/016905 which is hereby incorporated by reference herein in its entirety.

### TECHNICAL FIELD

The present invention relates to a package.

### BACKGROUND ART

There is a known sterile sheath for packaging a surgical instrument in a sterile state (see, for example, PTL 1).

This sterile sheath enables the packaging and transport of a surgical instrument in a sterile state, and when a system body of a surgical system and the surgical instrument are to be electrically connected to each other, connection is possible between electrical contacts of connectors provided on the system body or the surgical instrument by puncturing the sterile sheath with at least one of the connectors.

### CITATION LIST

### Patent Literature

### {Patent Literature 1} Japanese Translation of PCT International Application, Publication No. 2016-514562

### SUMMARY OF INVENTION

According to an aspect of the invention, a package is composed of a resin molded body that packages a manipulator of a medical manipulator system, the medical manipulator system including a base member having a connection target portion on an upper surface, and the manipulator having a connecting portion detachably connected to the connection target portion. The package includes a cover portion having a size capable of covering at least a portion of the upper surface of the base member in a state where the manipulator has been removed, and an opening forming portion having easy-cut lines provided in a portion of the cover portion. The opening forming portion is configured such that by cutting the opening forming portion along the easy-cut lines, it is possible to form, at a position that corresponds to the connection target portion when the cover portion is disposed at a position covering the upper surface, an opening portion that penetrates a predetermined region including the connection target portion in a plate thickness direction and wall portions that rise upward from an edge of the opening portion.

### BRIEF DESCRIPTION OF DRAWINGS

FIG. 1 is a perspective view illustrating an example of a medical manipulator system provided with a manipulator packaged with a package according to an embodiment of the present invention.

FIG. 2 is a perspective view illustrating an example of shafts of a drive portion provided at a base end of the manipulator of the medical manipulator system in FIG. 1.

FIG. 3 is a perspective view illustrating an example of shafts of a slider on a base member side on which the drive portion in FIG. 2 is to be fitted.

FIG. 4 is an exploded perspective view illustrating a state in which the manipulator is packaged with the package according to the embodiment of the present invention.

FIG. 5 is a perspective view illustrating a state in which opening portions and wall portions are formed in a lid member of the package in FIG. 4.

FIG. 6 is a perspective view illustrating a state where the base member is covered by the package in FIG. 5 and the drive portions of manipulators are connected to sliders exposed in the opening portions.

FIG. 7 is a plan view illustrating an example of an opening forming portion provided in the package in FIG. 4.

FIG. 8 is a partial longitudinal sectional view illustrating a modification of the package in FIG. 4.

FIG. 9 is a plan view illustrating a modification of the opening forming portion in FIG. 7.

FIG. 10 is a perspective view illustrating a modification of the lid member in FIG. 5 in which opening portions and wall portions are formed by the opening forming portion in FIG. 9.

FIG. 11 is a longitudinal sectional view illustrating a modification of the state in which an upper surface of the base member is covered with the lid member in FIG. 10.

FIG. 12 is a partial longitudinal sectional view illustrating another modification of the package in FIG. 4.

FIG. 13 is a partial longitudinal cross-sectional view illustrating a case where a container member of the package in FIG. 4 is used as a drip-proof member.

### DESCRIPTION OF EMBODIMENTS

A package 1 according to an embodiment of this invention is described below with reference to the drawings.

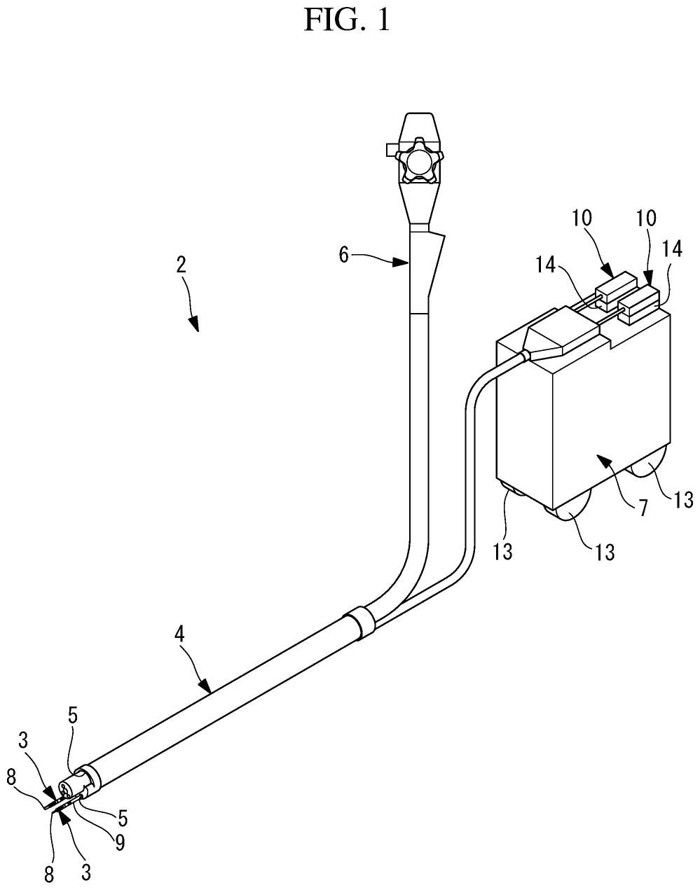

The package 1 according to the present embodiment packages a manipulator 3 of a medical manipulator system 2 illustrated in FIG. 1 for transport.

The medical manipulator system 2 includes an overtube 4, an endoscope 6 and two manipulators 3 that are respectively inserted into a lumen 5 of the overtube 4, and a base member 7 to which a base end of each of the manipulators 3 is detachably attached.

Each of the manipulators 3 includes an insertion portion 9 that is elongated and that has joints and an end effector 8 at a distal end, and a drive portion 10 provided at a base end of the insertion portion 9. Although, in the example illustrated in FIG. 1, the insertion portion 9 is a flexible insertion portion 9 having flexibility, the insertion portion 9 is not limited to this, and may be a rigid insertion portion 9.

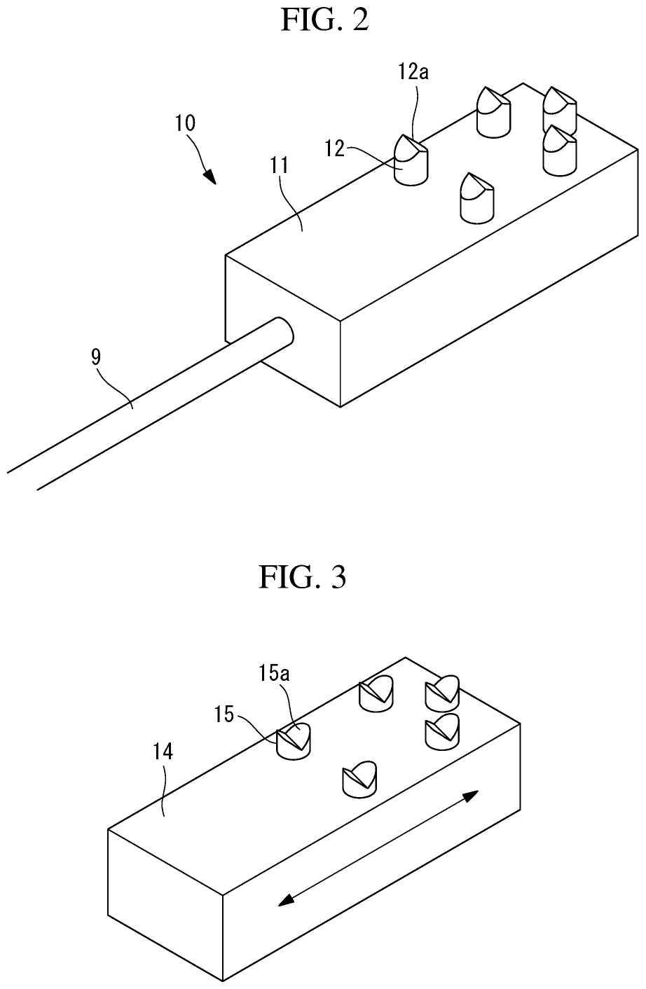

As illustrated in FIG. 2, the drive portion 10 includes a plurality of shafts (connecting portion) 12 protruding from one surface (a surface which is a lower surface in use in FIG. 1) of a case 11 having a substantially rectangular parallelepiped shape. A power transmission mechanism (not illustrated) is provided in the case 11, and a rotational force applied to the shafts 12 can be converted to tension of wires (not illustrated) disposed in the insertion portion 9 to operate the distal-end joints and the end effector 8. The distal end of each of the shafts 12 is provided with a wedge portion 12a that is tapered toward the distal end.

As illustrated in FIG. 1, the base member 7 is formed as a movable wagon having locking casters 13 on a bottom surface of the base member 7, and is provided with, on an upper surface thereof, two sliders 14 to be moved in one horizontal direction along the upper surface. As illustrated in FIG. 3, on each of the sliders 14, a plurality of shafts 15 are disposed facing upward, each of the shafts 15 being provided with a connector (connection target portion) 15a having a V groove complementary to the wedge portion 12a provided at each of the distal ends of the plurality of shafts 12 of the drive portion 10.

Each of the shafts 15 provided on the slider 14 is provided so as to be rotatable by a motor (not illustrated) disposed in the slider 14. In the state where the wedge portion 12a of the shaft 12 of the drive portion 10 is engaged with the connector 15a of the shaft 15 of the slider 14, by rotating the shaft 15 by rotation of the motor, the shaft 12 on the drive portion 10 side connected to the connector 15a is rotated, and a pulling force is generated on a wire corresponding to the shaft 12 so that a corresponding joint or the end effector 8 can be operated.

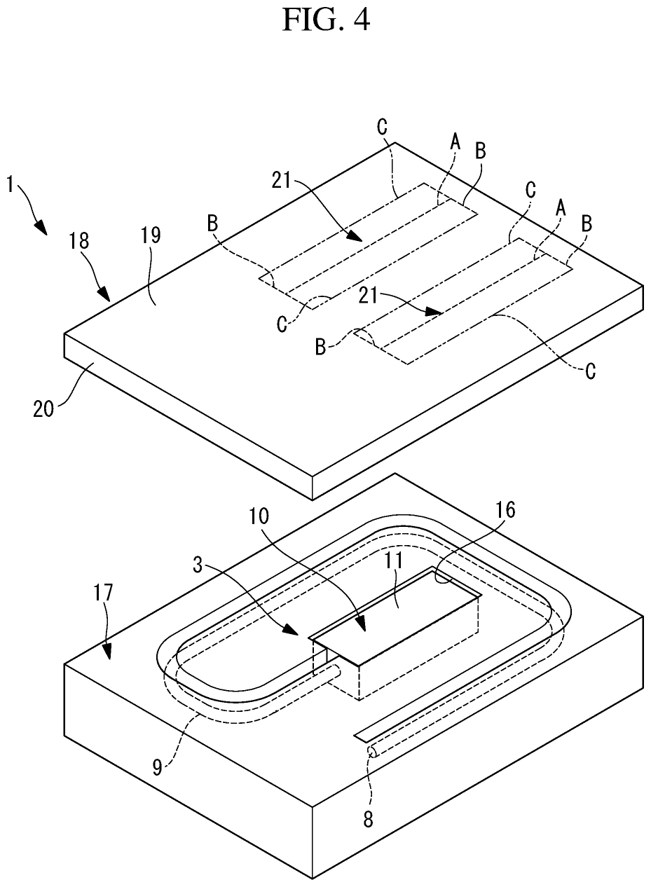

The package 1 according to the present embodiment, as illustrated in FIG. 4, includes a container member 17 having a recessed portion 16 that houses the manipulator 3 and a lid member (cover portion) 18 that packages the manipulator 3 housed in the recessed portion 16 in a sealed state by being combined with the container member 17. The container member 17 and the lid member 18 are respectively formed of thin resin molded bodies and have elasticity such that the container member 17 and the lid member 18 are deformable by an external force, but maintain a certain form as long as an external force is not applied thereto.

The package 1 according to the present embodiment is particularly characterized by the lid member 18. The lid member 18 is provided with a flat portion 19 and an edge 20 that fits to the outer surface of the container member 17 provided around the flat portion 19, as illustrated in FIG. 4. The lid member 18 has a size capable of covering the upper surface of the base member 7 of the medical manipulator system 2, and when the lid member 18 is arranged to cover the upper surface of the base member 7, opening forming portions 21 are provided in regions including the operating range of the connectors 15a, the operating range being realized by the sliders 14 provided on the base member 7.

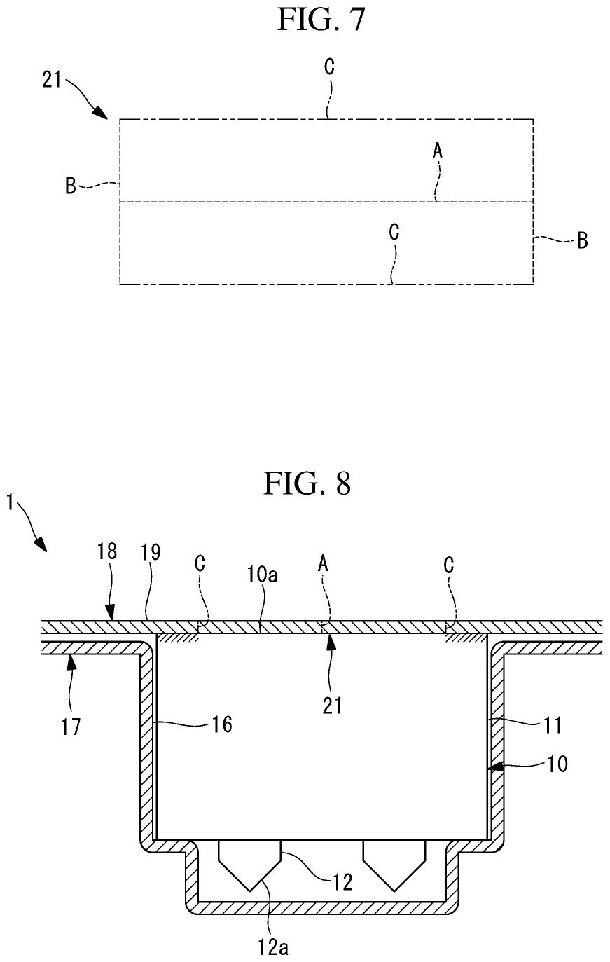

Each of the opening forming portions 21, as illustrated in FIG. 4, is provided with perforation lines (easy-cut lines) illustrated by broken lines for forming an opening portion 22 in a region including the operating range of the connectors 15a. The perforation lines, for example, as illustrated in FIG. 7, include a first perforation line A provided in a straight line along the longitudinal direction of a portion to be the opening portion 22 at the center in the width direction of the portion to be the opening portion 22 and second perforation lines B provided on both ends of the first perforation line A and orthogonal to the first perforation line A along the entire length in the width direction of the portion to be the opening portion 22.

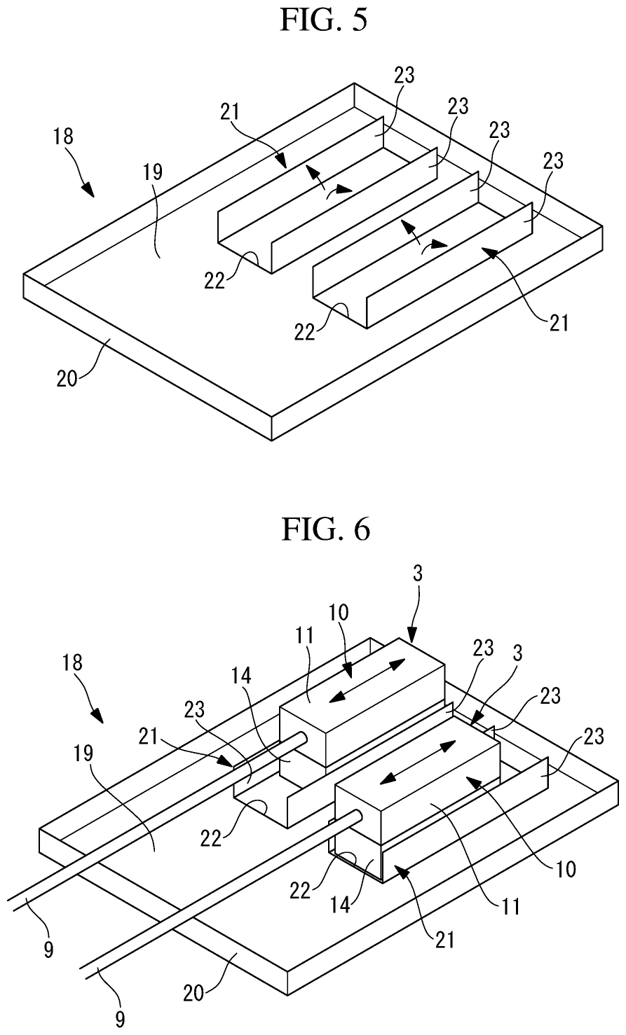

Then, after these perforation lines A and B have been cut, the portion to be the opening portion 22 is bent along fold lines C parallel to the first perforation line A and, as illustrated in FIG. 5, wall portions 23 rising from the edge of the opening portion 22 along the longitudinal direction are formed on both sides in the width direction of the opening portion 22.

In addition, in the package 1 according to this embodiment, in the packaged state in which the manipulator 3 is housed in the recessed portion 16 of the container member 17 and the lid member 18 is combined therewith, the opening forming portion 21 is disposed so that the manipulator 3 does not abut against the inside of the opening forming portion 21 provided in the lid member 18. For example, in the case where the drive portion 10 has a flat surface 10a, in addition to disposing the opening forming portion 21 at a position away from the drive portion 10, as illustrated in FIG. 8, the opening forming portion 21 may be disposed on the inside of the flat surface 10a of the drive portion 10.

When the lid member 18, which is flat, is pushed by the flat surface 10a of the drive portion 10, the peripheral portion (the hatched portion in the drawing) of the flat surface 10a abuts against the lid member 18, and the lid member 18 is not pressed only by the center portion of the flat surface 10a. Therefore, by arranging the opening forming portion 21 on the inside of the flat surface 10a, no force is applied to the opening forming portion 21 from the drive portion 10, and it is possible to prevent, in advance, inconvenient tearing of the first and second perforation lines A and B during transport.

The operation of the package 1 according to the present embodiment configured as described above will be described below.

In order to transport the manipulator 3 using the package 1 according to the present embodiment, the drive portion 10 and the insertion portion 9 of the manipulator 3 are housed in the recessed portion 16 of the container member 17 and, the manipulator 3 is packaged in a sealed state by combining the lid member 18 with the container member 17.

At this time, a surface on the recessed portion 16 side of the container member 17 disposed on the manipulator 3 side and a surface of the lid member 18 disposed on the manipulator 3 side are sterilized, and the manipulator 3 is maintained in a sterile state during transport.

In addition, by housing the manipulator 3 in the container member 17 formed of a thin resin molded body, an impact during transport is suppressed by the elastic deformation of the container member 17, and the manipulator 3 can be transported without being subjected to an impact.

After the manipulator 3 has been transported to an operating room, and after removing the lid member 18 from the container member 17 and cutting the lid member 18 along the first perforation line A and the second perforation lines B provided on the lid member 18, by folding the cut portions along the fold lines C to the sterilized surface side, the opening portion 22 and the wall portions 23 are formed as illustrated in FIG. 5.

By arranging the lid member 18 thus processed on the upper surface of the base member 7, the sliders 14 can be exposed from the opening portions 22 and other portions can be covered.

Then, the wedge portions 12a of the shafts 12 of the drive portion 10 of the manipulator 3 are made to coincide with corresponding connectors 15a of the shafts 15 of the slider 14 exposed from the opening portion 22, and by fixing the drive portion 10 to the slider 14, the setting is completed as illustrated in FIG. 6.

Thereby, since the lid member 18 covers the entire upper surface of the base member 7, even if a liquid such as bodily fluid is scattered, it can be prevented from adhering to the base member 7. In addition, since the edge 20 provided on the lid member 18 extends upward to constitute a peripheral wall portion, it is possible to keep liquid that has splashed and adhered to the lid member 18 from flowing out to the outside.

Furthermore, since the size of the opening portion 22 is set so as to cover the operating range of the slider 14, as illustrated by the arrows in FIG. 6, even if the slider 14 operates, the lid member 18 does not inhibit the operation of the slider 14.

In addition, since the wall portions 23 rising from the edges of the opening portion 22 by bending the opening forming portion 21 are provided, scattered bodily fluid or the like can be prevented from flowing into the opening portion 22 by the wall portion 23.

That is, according to the package 1 according to this embodiment, the manipulator 3 can be packaged and transported while maintaining a sterile state, and the soundness of the manipulator 3 can be maintained by suppressing any impact applied to the manipulator 3 during transport. In addition, after transport, the lid member 18 removed from the container member 17 is turned over, the first perforation line A and the second perforation lines B of the opening forming portion 21 are cut to form the opening portion 22, the wall portions 23 are formed by bending the periphery of the opening portions 22 along the fold lines C, and the lid member 18 can be used as a drip-proof member which covers the upper surface of the base member 7 to which the drive portion 10 of the manipulator 3 is attached.

As a result, the opening portion 22 formed to have a size that covers the operating range of the slider 14 prevents dripping on the upper surface of the base member 7 without obstructing the operation of the manipulator 3 by the slider 14, and the wall portions 23 rising at the edge of the opening portion 22 can improve the effect of preventing the liquid from entering the opening portion 22. In particular, since the package 1, which becomes unnecessary after transportation, is reused, there is no need to prepare a drip-proof member separately, which is advantageous in that the cost can be reduced.

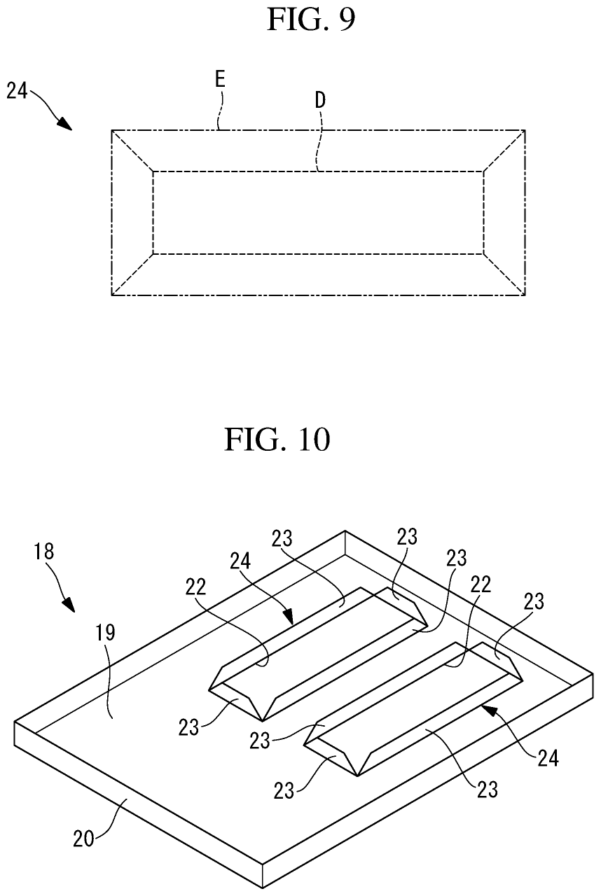

Further, in the present embodiment, the opening forming portion 21 is formed by cutting the first and second perforation lines A and B illustrated in FIG. 7 and bending along the fold lines C to form the opening portion 22 and the wall portions 23; however, an opening forming portion 24 as illustrated in FIG. 9 may be employed. According to the opening forming portion 24, processing can be performed such that a center portion is cut out along the perforation lines (easy-cut lines) D, and, as illustrated in FIG. 10, by bending in four directions along fold lines E, wall portions 23 are formed over the entire circumference of the opening portion 22. Thereby, the inflow of liquid to the opening portion 22 can be more effectively blocked.

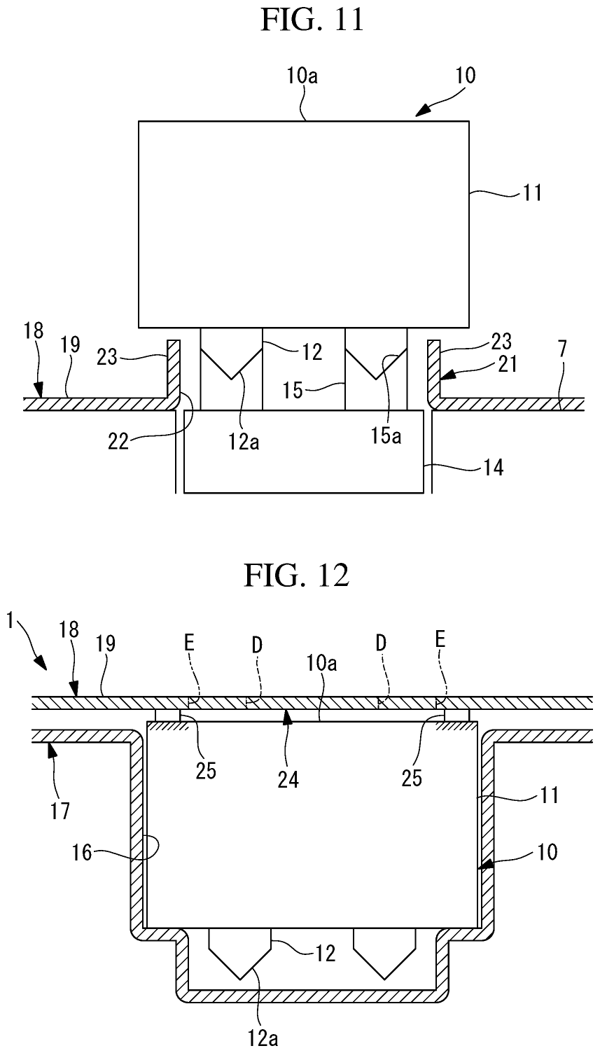

In addition, in the present embodiment, the slider 14 on the base member 7 is made to penetrate through the opening portion 22 formed in the opening forming portion 21; however, depending on the state of the slider 14 in the base member 7, as illustrated in FIG. 11, in some cases, the upper surface of the base member 7 and the upper surface of the slider 14 may be flush with each other. In such a case, the wall portions 23 can be disposed below the drive portion 10, and the inflow of liquid from above can be more effectively prevented.

In addition, as illustrated in FIG. 12, when the lid member 18 is combined with the container member 17, protrusions (pressing portions) 25 that abut against the drive portion 10 of the manipulator 3 housed in the recessed portion 16 of the container member 17 may be provided on the surface of the lid member 18. In this case, as illustrated in FIG. 12, it is preferable to dispose the protrusions 25 outside the opening forming portion 24.

By doing so, even if the manipulator 3, which is packaged, vibrates during transport of the manipulator 3, the lid member 18 can receive, at the positions of the protrusions 25, vibrations from the manipulator 3, and it is possible to avoid the inconvenience caused when a force is applied to the perforation lines D during transport, causing the perforation lines D to be torn.

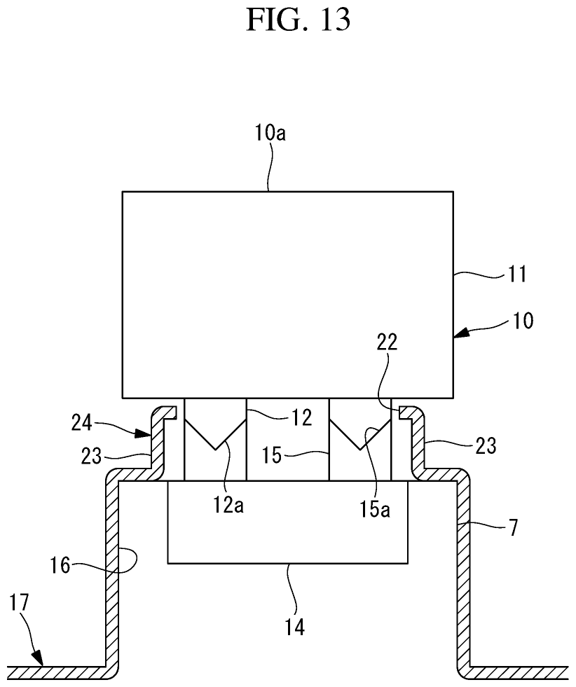

In addition, in this embodiment, the lid member 18 is provided with the opening forming portions 21 and 24 and is disposed so as to cover the upper surface of the base member 7; however, instead of this, depending on the shape of the base member side, as illustrated in FIG. 13, the container member 17 may be provided with the opening forming portion 24 and cut along the perforation lines D to form the opening portion 22 and may be used as a drip-proof member. In the example illustrated in FIG. 13, just by forming the opening portion 22 by cutting out the bottom surface of the recessed portion 16 of the container member 17 that has housed the drive portion 10 of the manipulator 3 along the fold lines (easy-cut lines) E as illustrated in FIG. 12, it is possible to form a drip-proof member having the wall portions 23 over the entire circumference of the opening portion 22.

In addition, in the present embodiment, the lid member 18 or the container member 17 is reused as a drip-proof member; however, a package 1 in which the lid member 18 and the container member 17 are connected may be used.

The above-described embodiment leads to the following invention.

According to an aspect of the invention, a package is composed of a resin molded body that packages a manipulator of a medical manipulator system, the medical manipulator system including a base member having a connection target portion on an upper surface, and the manipulator having a connecting portion detachably connected to the connection target portion. The package includes a cover portion having a size capable of covering at least a portion of the upper surface of the base member in a state where the manipulator has been removed, and an opening forming portion having easy-cut lines provided in a portion of the cover portion. The opening forming portion is configured such that by cutting the opening forming portion along the easy-cut lines, it is possible to form, at a position that corresponds to the connection target portion when the cover portion is disposed at a position covering the upper surface, an opening portion that penetrates a predetermined region including the connection target portion in a plate thickness direction and wall portions that rise upward from an edge of the opening portion.

According to this aspect, by packaging the manipulator in a package formed of a resin molded body, the manipulator is fixed by the package so as not to move, and the elasticity of the package can reduce an impact on the manipulator.

Then, after removing the manipulator from the package, by cutting along the easy-cut lines of the opening forming portion provided in the cover portion and bending portions adjacent to the easy-cut lines, an opening portion penetrating in the thickness direction is formed in a portion of the cover portion.

Therefore, when the cover portion of the package formed in this way is arranged so that the upper surface of the base member is covered by the cover portion, the opening portion provided in the cover portion is aligned with the connection target portion provided on the upper surface of the base member, a region including the connection target portion is exposed, and other portions are covered by the cover portion.

As a result, the connecting portion of the manipulator can be connected to the connection target portion of the base member through the opening portion, and wall portions rising from the edge of the opening portion are formed.

That is, the wall portions can prevent liquid from entering the opening portion from the outside, a drip-proof effect can be improved and the base member can be protected.

In the above-mentioned aspect, the opening forming portion may be configured such that, by bending portions adjacent to the easy-cut lines after the opening forming portion has been cut along the easy-cut lines, it is possible to form the wall portions rising upward from the edge of the opening portion.

Thus, by cutting along the easy-cut lines of the opening forming portion provided in the cover portion and bending the portions adjacent to the easy-cut lines, the opening portion and the wall portions penetrating in the thickness direction to a portion of the cover portion can be easily formed.

In the above aspect, the connection target portion may be provided so as to be movable in a horizontal direction with respect to the base member, and the easy-cut lines may be provided such that it is possible to form the opening portion in a predetermined region including the movement range of the connection target portion.

Thus, even if the connecting portion of the manipulator is connected to the connection target portion of the base member and the connection target portion is moved in the horizontal direction, because the connection target portion moves within the opening portion, it is possible to maintain the covered state of the base member by the cover portion.

In addition, in the above aspect, the opening forming portion may be configured such that it is possible to form the wall portions extending along a movement direction of the connection target portion on both sides of the opening portion.

Thus, by connecting the connecting portion of the manipulator to the connection target portion of the base member, and moving the connection target portion in the horizontal direction, the connection target portion moves within the opening portion. Since the wall portions extend along the movement direction of the connection target portion at the edge of the opening portion, the wall portions can maintain the effect of preventing liquid from entering the opening portion even if the connection target portion moves.

In addition, in the above-mentioned aspect, the above-mentioned opening forming portion may be configured such that it is possible to form the wall portions around the entire periphery of the opening portion.

Thus, by connecting the connecting portion of the manipulator to the connection target portion of the base member, and moving the connection target portion in the horizontal direction, the connection target portion moves within the opening portion. Since the wall portions extend all the way around the edge of the opening portion, it is possible to maintain the effect of preventing liquid from entering the opening portion by the wall portions even if the connection target portion moves.

In addition, in the above aspect, pressing portions that press the manipulator in a state where the manipulator is packaged may be provided only on the outside of the opening forming portion.

Thus, there are no pressing portions that press the manipulator on the inside of the opening forming portion, and, for example, during transport of the packaged manipulator, the manipulator can be maintained in the packaged state by preventing a force capable of breaking the easy-cut lines of the opening forming portion from acting on the inside of the opening forming portion from the manipulator.

In addition, in the above aspect, the pressing portions may include protrusions that protrude in a thickness direction with respect to the opening forming portion.

Thus, because the packaged manipulator is pressed only by the pressing portions formed of the protrusions that protrude on the outside of the opening forming portion, there is no pressing portion that presses the manipulator on the inside of the opening forming portion, and during transport of the packaged manipulator, the manipulator can be maintained in the packaged state by preventing a force capable of breaking the easy-cut lines of the opening forming portion from acting on the inside of the opening forming portion from the manipulator.

In addition, in the above aspect, the wall portions may be configured to be formed by bending the opening forming portion to a side where the manipulator is disposed at the time of packaging.

Thus, because the side on which the manipulator is disposed in the packaged state is subjected to a sterilization treatment, the manipulator removed from the package can be disposed on the sterilized side of the cover portion covering the upper surface of the base member.

### REFERENCE SIGNS LIST

1 package
2 medical manipulator system
3 manipulator
7 base member
12 shaft
15a connector (connection target portion)
18 lid member (cover portion)
21, 24 opening forming portion
22 opening portion
23 wall portion
25 protrusion (pressing portion)
A first perforation line (easy-cut line)
B second perforation lines (easy-cut lines)
D perforation lines (easy-cut lines)
E fold lines (easy-cut lines)

1. A package, wherein
the package is composed of a resin molded body that packages a manipulator of a medical manipulator system, the medical manipulator system including a base member having a connection target portion on an upper surface, and the manipulator having a connecting portion detachably connected to the connection target portion,
the package comprises a cover portion having a size capable of covering at least a portion of the upper surface of the base member in a state where the manipulator has been removed, and an opening forming portion having easy-cut lines provided in a portion of the cover portion, and
the opening forming portion is configured such that by cutting the opening forming portion along the easy-cut lines, it is possible to form, at a position that corresponds to the connection target portion when the covering portion is disposed at a position covering the upper surface, an opening portion that penetrates a predetermined region including the connection target portion in a plate thickness direction and wall portions that rise upward from an edge of the opening portion.

2. The package according to claim 1, wherein the opening forming portion is configured such that, by bending portions adjacent to the easy-cut lines after the opening forming portion has been cut along the easy-cut lines, it is possible to form the wall portions rising upward from the edge of the opening portion.

3. The package according to claim 1, wherein the connection target portion is provided so as to be movable in a horizontal direction with respect to the base member, and
the easy-cut lines are provided such that it is possible to form the opening portion in a predetermined region including a movement range of the connection target portion.

4. The package according to claim 3, wherein the opening forming portion is configured such that it is possible to form the wall portions extending along a movement direction of the connection target portion on both sides of the opening portion.

5. The package according to claim 3, wherein the opening forming portion is configured such that it is possible to form the wall portions around the entire periphery of the opening portion.

6. The package according to claim 1, wherein pressing portions that press the manipulator in a state where the manipulator is packaged are provided only on the outside of the opening forming portion.

7. The packaging body according to claim 6, wherein the pressing portions include protrusions that protrude in a thickness direction with respect to the opening forming portion.

8. The package according to claim 1, wherein the wall portions are configured to be formed by bending the opening forming portion to a side where the manipulator is disposed at the time of packaging.
